(19) United States
(12) Patent Application Publication
(10) Pub. No.: US 2007/0196851 A1
(43) Pub. Date: Aug. 23, 2007

(54) System and method for selecting differentially dependent genes from gene expression data

(76) Inventors: Andrei Yakovlev, Pittsford, NY (US); Lev Klebanov, Prague (CZ); Xing Qui, Rochester, NY (US)

Correspondence Address: BLANK ROME LLP, 600 NEW HAMPSHIRE AVENUE, N.W., WASHINGTON, DC, 20037, US

(21) Appl. No.: 11/705,034

(22) Filed: Feb. 12, 2007

Related U.S. Application Data
(60) Provisional application No. 60/772,876, filed on Feb. 14, 2006.

(51) Int. Cl. C12Q 1/68; G06F 19/00

(52) U.S. Cl. 435/6; 702/20

(57) ABSTRACT

Dependence between expression levels of different genes is identified through a multivariate method of statistical analysis of single color microarray gene expression data. An algorithm is applied to gene data to identify those genes that are likely to change their relationship with all other genes, and select such genes for further investigation. If genes change their relationship to other genes from phenotype to phenotype, they are treated as likely to change their relationship with one another.

### REFERENCE TO RELATED APPLICATIONS

The present application claims the benefit of U.S. Provisional Patent Application No. 60/772,876, filed Feb. 14, 2006. Related information is disclosed in U.S. patent application Ser. No. 11/593,635, filed Nov. 7, 2006. The disclosures of both of the above applications are hereby incorporated by reference in their entireties into the present application.

### STATEMENT REGARDING FEDERALLY SPONSERED RESEARCH OR DEVELOPMENT

The U.S. Government has a paid-up license in this invention and the right in limited circumstances to require the patent owner to license others on reasonable terms as provided for by the terms of Grant GM075299 awarded by NIH/NIGMS.

### FIELD OF THE INVENTION

This invention relates generally to the field of identifying relationships between genes using statistical analysis.

### BACKGROUND OF THE INVENTION

It has become common practice to use microarray technology for finding “interesting” genes by comparing two or more different phenotypes. Modern methods of microarray data analysis typically employ two-sample statistical tests combined with multiple testing procedures to control a Type 1 error rate.

Such methods are often designed to select those genes that display the most pronounced differential expression. Once the list of genes showing statistically significant differential expression has been generated, these genes are often ranked using purely statistical criteria and this ranking is intended to reflect their relative importance. Typically, a certain number of genes with the smallest p-values are finally selected from the list of all “significant” ones. While most biologists recognize that the magnitude of differential expression does not necessarily indicate biological significance, this approach is one that has been conventionally used for initial prioritizing of candidate genes.

From a biological perspective, the above-described paradigm falls far short of being a perfectly valid approach. Even a very small change in expression of a particular gene may have dramatic physiological consequences if the protein encoded by this gene plays a catalytic role in a specific cell function.

The inventors have determined that downstream genes may amplify the signal produced by a gene that is truly interesting, thereby increasing the chance that the downstream genes will be selected by formal statistical methods, rather than the gene of greater actual interest. For a regulatory gene, however, the inventors have determined that the chance of being selected by such methods often diminishes as one keeps hunting for downstream genes which tend to show much bigger changes in their expression. As a result, the final list of candidates may be enriched with many effector genes that do little to elucidate more fundamental mechanisms of biological processes.

There are two natural ways to remedy the situation. One is to use bioinformatics tools that utilize prior biological knowledge, such as partially known pathways, for prioritization of candidate genes. This approach is now routinely used in biological studies and there are ongoing efforts to enrich it with specially designed algorithms such as the well-known Gene Set Enrichment Analysis (GSEA).

However, the significance analysis based on the GSEA is essentially confirmatory and does not offer an alternative to the much needed exploratory tools. The current biological knowledge is still very limited and sometimes inaccurate. Gene annotations do not provide an unambiguous guide to selection of individual genes, let alone gene combinations. Another way is to extract more information from microarray data in the course of exploratory analysis by pertinent statistical methods, which is the general thrust of the present invention.

Recent years have seen a growing interest in correlations between gene expression levels in statistical methodologies for microarray analysis. For example, see the following articles: Xiao Y., Frisina R., Gordon A., Klebanov L., Yakovlev A. (2004) “Multivariate search for differentially expressed gene combinations,” BMC Bioinformatics 5: # 164; Dettling M., Gabrielson E., Parmigiani G. (2005) “Searching for differentially expressed gene combinations,” http://www.bepress.com/ jhubiostat/paper77; Qiu X., and Brooks A. I., Klebanov L., Yakovlev A. (2005) “The effects of normalization on the correlation structure of microarray data,” BMC Bioinformatics 6: # 120.

### SUMMARY OF THE INVENTION

The inventors have determined that there is a need to invoke additional information for each gene by analyzing dependence between gene expressions.

It is to be understood that both the following summary and the detailed description are exemplary and explanatory and are intended to provide further explanation of the invention as claimed. Neither the summary nor the description that follows is intended to define or limit the scope of the invention to the particular features mentioned in the summary or in the description.

In the main embodiment, an algorithm is provided to facilitate identification of genes for further consideration on the basis that they are likely to change their relationships with other genes. In an exemplary embodiment of the algorithm, (1) a kernel L(x,y) is selected. (2) Subsamples are drawn randomly from the original collection of sample vectors reporting expression levels of the genes for each of two groups. (3) Vectors R₁ [symbol] (i) and R₁ [symbol] (i) are calculated for the (empirical) correlations between the i th coordinate of the vector X and all of its other coordinates. (4) Steps 2 and 3 are repeated to obtain S₁(i)=R₁ [symbol] (i), . . . , Sₖ(i)=Rₖ [symbol] (i), and T₁(i)=R₁ [symbol] (i), . . . , Tₖ(i)=Rₖ [symbol] (i). (5) Steps 2 through 4 are repeated two more times to obtain S₁′(i), . . . , Sₖ′(i), Tₖ′(i), . . . , Tₖ′(i), S₁″(i), . . . , Sₖ″(i), and Tₖ″(i), . . . , Tₖ″(i). (6) Latent variables Uⱼ(i)=L(Sⱼ(i), Tⱼ(i))−L(Sⱼ(i), Sⱼ(i)) and Vⱼ(i)=L(Tⱼ′(i), Tⱼ″(i))−L(Sⱼ″(i), Tⱼ″(i)), j=1, . . . , k are calculated. (7) A distribution-free two-sample univariate test is applied to the samples Uⱼ (i) and Vⱼ (i), j =1, . . . ,k, to obtain a corresponding p-value. (8) Steps 2 through 6 are repeated to obtain all unadjusted p-values for the hypotheses Hᵢ, and the resultant p-values are adjusted for multiple testing.

Additional features and advantages of the invention will be set forth in the description which follows, and in part will be apparent from the description, or may be learned by practice of the invention. The advantages of the invention will be realized and attained by the structure particularly pointed out in the written description and claims hereof as well as the appended drawings.

The following paper is hereby incorporated by reference in its entirety into the present disclosure: Klebanov, L., Jordan, C., and Yakovlev, A., “A new type of stochastic dependence revealed in gene expression data,” Statistical Applications in Genetics and Molecular Biology, 2006, vol. 5, Article 7.

### BRIEF DESCRIPTION OF THE DRAWINGS

The accompanying drawings, which are incorporated herein and form a part of the specification, illustrate various exemplary embodiments of the present invention and, together with the description, further serve to explain various principles and to enable a person skilled in the pertinent art to make and use the invention.

FIG. 1 is a block schematic diagram showing an example of a computer system that can be used to implement some embodiments.

FIG. 2 is a flow chart of an exemplary algorithm useful in identifying genes that are likely to change their relationship with other genes.

The present invention will be described with reference to the accompanying drawings. In the drawings, some like reference numbers indicate identical or functionally similar elements. Additionally, the left-most digit(s) of most reference numbers may identify the drawing in which the reference numbers first appear.

### DETAILED DESCRIPTION OF THE PREFERRED EMBODIMENT

The present invention will now be explained in terms of an exemplary embodiment. This specification discloses one or more embodiments that incorporate the features of this invention. The disclosure herein will provide examples of embodiments, including examples of data analysis from which those skilled in the art will appreciate various novel approaches and features developed by the inventors.

These various novel approaches and features, as they may appear herein, may be used individually, or in combination with each other as desired.

In particular, the embodiment(s) described, and references in the specification to “one embodiment”, “an embodiment”, “an example embodiment”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, persons skilled in the art may effect such feature, structure, or characteristic in connection with other embodiments whether or not explicitly described.

Embodiments of the invention may be implemented in hardware, firmware, software, or any combination thereof, or may be implemented without automated computing equipment. Embodiments of the invention may also be implemented as instructions stored on a machine-readable medium, which may be read and executed by one or more processors. A machine-readable medium may include any mechanism for storing or transmitting information in a form readable by a machine (e.g. a computing device). For example, a machine-readable medium may include read only memory (ROM); random access memory (RAM); hardware memory in PDAs, mobile telephones, and other portable devices; magnetic disk storage media; optical storage media; flash memory devices; electrical, optical, acoustical, or other forms of propagated signals (e.g. carrier waves, infrared signals, digital signals, analog signals, etc.), and others. Further, firmware, software, routines, instructions, may be described herein as performing certain actions. However, it should be appreciated that such descriptions are merely for convenience and that such actions in fact result from computing devices, processors, controllers or other devices executing the firmware, software, routines, instructions, etc.

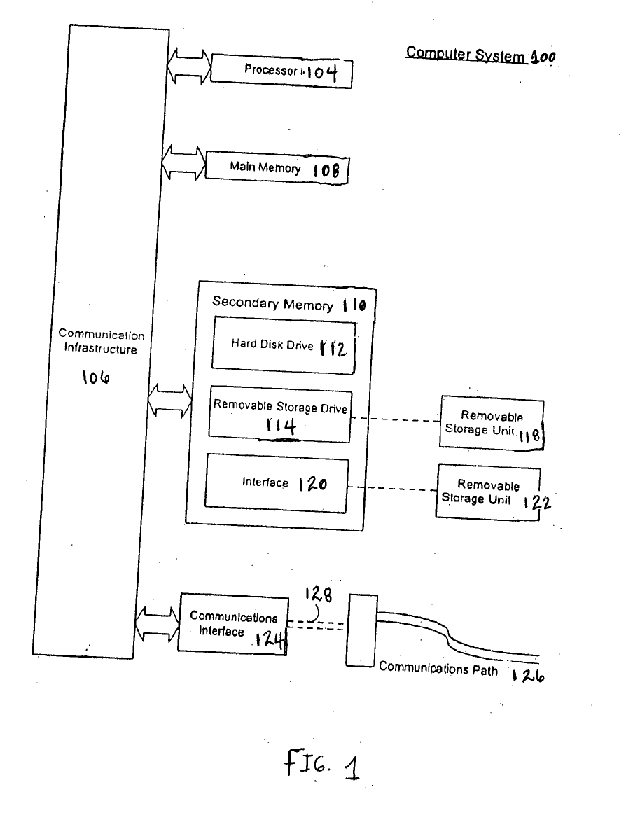

The following description of a general purpose computer system, such as a PC system, is provided as a non-limiting example of systems on which the disclosed analysis can be performed. In particular, the methods disclosed herein can be performed manually, implemented in hardware, or implemented as a combination of software and hardware. Consequently, desired features of the invention may be implemented in the environment of a computer system or other processing system. An example of such a computer system 100 is shown in FIG. 1. The computer system 100 includes one or more processors, such as processor 104. Processor 104 can be a special purpose or a general purpose digital signal processor. The processor 104 is connected to a communication infrastructure 106 (for example, a bus or network). Various software implementations are described in terms of this exemplary computer system. After reading this description, it will become apparent to a person skilled in the relevant art how to implement the invention using other computer systems and/or computer architectures.

Computer system 100 also includes a main memory 105, preferably random access memory (RAM), and may also include a secondary memory 110. The secondary memory 110 may include, for example, a hard disk drive 112, and/or a RAID array 116, and/or a removable storage drive 114, representing a floppy disk drive, a magnetic tape drive, an optical disk drive, etc. The removable storage drive 114 reads from and/or writes to a removable storage unit 118 in a well known manner. Removable storage unit 118, represents a floppy disk, magnetic tape, optical disk, etc. As will be appreciated, the removable storage unit 118 includes a computer usable storage medium having stored therein computer software and/or data.

In alternative implementations, secondary memory 110 may include other similar means for allowing computer programs or other instructions to be loaded into computer system 100. Such means may include, for example, a removable storage unit 122 and an interface 120. Examples of such means may include a program cartridge and cartridge interface (such as that found in video game devices), a removable memory chip (such as an EPROM, or PROM) and associated socket, and other removable storage units 122 and interfaces 120 which allow software and data to be transferred from the removable storage unit 122 to computer system 100.

Computer system 100 may also include a communications interface 124. Communications interface 124 allows software and data to be transferred between computer system 100 and external devices. Examples of communications interface 124 may include a modem, a network interface (such as an Ethernet card), a communications port, a PCMCIA slot and card, etc. Software and data transferred via communications interface 124 are in the form of signals 128 which may be electronic, electromagnetic, optical or other signals capable of being received by communications interface 124. These signals 128 are provided to communications interface 124 via a communications path 126. Communications path 126 carries signals 128 and may be implemented using wire or cable, fiber optics, a phone line, a cellular phone link, an RF link and other communications channels.

The terms “computer program medium” and “computer usable medium” are used herein to generally refer to media such as removable storage drive 114, a hard disk installed in hard disk drive 112, and signals 128. These computer program products are means for providing software to computer system 100.

Computer programs (also called computer control logic) are stored in main memory 108 and/or secondary memory 110. Computer programs may also be received via communications interface 124. Such computer programs, when executed, enable the computer system 100 to implement the present invention as discussed herein. In particular, the computer programs, when executed, enable the processor 104 to implement the processes of the present invention. Where the invention is implemented using software, the software may be stored in a computer program product and loaded into computer system 100 using raid array 116, removable storage drive 114, hard drive 112 or communications interface 124.

In the preferred embodiment, a method is implemented that selects those genes that are most likely to change their relationship with other genes. Initially, this method will be explained by providing background information that is useful in understanding the disclosed method and its variations.

Microarray expression data on m distinct genes can be thought of as a sample of random vectors Z=(Z₁, . . . , Zₘ) with stochastically dependent components. The dimension of Z is typically very high relative to the number of observations (arrays). Suppose that sample observations are available on gene expression levels in two phenotypes (conditions) A and B. In the simplest case of equal sample sizes, the expression data are represented by two m×n matrices: Zᵢⱼ( [symbol] ) for condition [symbol] and Zᵢⱼ( [symbol] ) for condition [symbol] , i=1, . . . , m, j=1, . . . , n, where m is the number of genes and n is the number of arrays per condition. For the i th gene in a given phenotype, m−1 sample correlation coefficients rᵢₖ, k=1, . . . , m−1 can be computed for all pairs formed by this gene with all other genes. As a result, each gene will be characterized by an (m−1)-dimensional vector of correlation coefficients Rᵢⁿ=(rᵢₗ, . . . , rᵢ₍ₘ₋₁₎). For the i th gene in phenotype [symbol] , this vector may be denoted by Rᵢⁿ( [symbol] ). In like manner, the i th gene in phenotype [symbol] can be characterized by the correlation vector Rᵢⁿ( [symbol] ).

One objective in exemplary embodiments of this method is to test the statistical inference from microarray gene expression data by testing the hypothesis: the ith gene does not change its relationships with all other genes across the two conditions under study. This can be accomplished by comparing the two sample vectors Rᵢⁿ( [symbol] ) and Rᵢⁿ( [symbol] ) for each gene. To design a pertinent statistical test, the basic null hypothesis is formulated as Hᵢ: F_{R}_{ᵢ}(x)=G_{R}_{ᵢ}(x), where F_{R}_{ᵢ}(x) and G_{R}_{ᵢ}(x) are the true (m−1)-dimensional distributions of the vectors Rᵢⁿ( [symbol] ) and Rᵢⁿ( [symbol] ) associated with the i th gene. The hypothesis Hᵢ is more general than the hypothesis Hᵢ′: ρᵢ( [symbol] )=ρᵢ( [symbol] ), where ρᵢ( [symbol] ) and ρᵢ( [symbol] ) are the corresponding vectors of the true correlation coefficients. If rᵢₖ are unbiased estimators for the true correlation coefficients ρᵢₖ and the hypothesis Hᵢ is true, then the hypothesis Hᵢ is true as well. However, the converse is generally not true.

While the primary focus in this approach is on testing the hypothesis Hᵢ, it is also desirable to provide tools to make this test more sensitive to departures from Hᵢ′. A first expedient relates to the choice of a statistic to represent rᵢₖ. Denote the Pearson sample correlation coefficient for the i th gene in gene pair k by wᵢₖ and introduce Fisher's transformation score

$${r}_{ik} = \frac{1}{2} log \frac{1 + {w}_{ik}}{1 - {w}_{ik}} \qquad Equation ( 1 )$$

as a measure of correlation between the expression levels in this pair. It is well known that the statistic rᵢₖ has an asymptotic normal distribution with mean

$$\frac{1}{2} log [ ( 1 + {ρ}_{ik} ) / ( 1 - {ρ}_{ik} ) ]$$

and variance 1/\sqrt{n−3}. Note that the asymptotic variance does not depend on the unknown parameter ρᵢₖ, which property makes the marginal (for the k th gene pair formed by gene i) hypotheses Hᵢₖ and Hᵢₖ′ approximately equivalent in large samples. When working with finite samples, the bias of order 1/n in the estimator wᵢₖ can be removed by using a modification of the sample correlation coefficient proposed by Olkin and Pratt (1958).

A second expedient relates to the choice of a multivariate test-statistic to measure differences between F_{R}_{ᵢ}(x) and G_{R}_{ᵢ}(x) when designing a distribution-free statistical test for Hᵢ. As shown elsewhere in the present disclosure, a two-sample test-statistic can be selected that is predominantly sensitive to differences in marginal distributions when comparing the joint distributions F_{R}_{ᵢ}(x) and G_{R}_{ᵢ}(x). By combining the two expedients we make the hypotheses Hᵢ and Hᵢ′ approximately equivalent at least in large sample studies. While the hypothesis Hᵢ′ is easier to interpret in terms of the true correlation vectors, the more general hypothesis Hᵢ is still quite meaningful as a means of making inference about changes in the correlation structure of microarray data.

A multivariate distribution-free test for the hypothesis Hᵢ will now be described in more detail.

Let X=X₁, . . . , X_{d} and Y=Y₁, . . . , Y_{d} be two random vectors with probability distributions μ and ν, respectively, defined on the Euclidean space R^{d}. Let L(x, y) be a strictly negative definite kernel (see, Vakhaniya et al., 1987) defined for arbitrary d -dimensional vectors, that is is Σ_{i,j=1}^{s} L(xᵢ, xⱼ)hᵢhⱼ≦0 for any x₁, . . . , xₛ from R^{d} and any real numbers h₁, . . . hₛ, Σᵢ₌₁^{s} hᵢ=0, with equality if and only if all hᵢ=0. Introduce the following distance between μ and ν:

$$N ( μ , v ) = {[ \begin{matrix}2 {\int }_{{ℝ}^{d}} {\int }_{{ℝ}^{d}} L ( x , y ) ⅆ μ ( x ) ⅆ v ( y ) - \\ {\int }_{{ℝ}^{d}} {\int }_{{ℝ}^{d}} L ( x , y ) ⅆ μ ( x ) ⅆ μ ( y ) - \\ {\int }_{{ℝ}^{d}} {\int }_{{ℝ}^{d}} L ( x , y ) ⅆ v ( x ) ⅆ v ( y )\end{matrix} ]}^{1 / 2} . \qquad Equation ( 2 )$$

The distance N(μ,ν) was shown (Zinger et al., 1989) to be a metric in the space of all probability measures on R^{d}, so that the null hypothesis in two-sample comparisons can be formulated as H₀: N(μ, ν)=0. This metric has been used successfully for selecting differentially expressed genes and gene combinations in microarray data analysis (See Szabo et al., 2002, 2003; Xiao et al., 2004; Klebanov et al., 2005); it plays a useful role in the analysis that follows.

Assuming that

$${\int }_{{ℝ}^{d}} {\int }_{{ℝ}^{d}} L ( x , y ) ⅆ μ ( x ) ⅆ μ ( y ) < ∞ , \\ {\int }_{{ℝ}^{d}} {\int }_{{ℝ}^{d}} L ( x , y ) ⅆ v ( x ) ⅆ v ( y ) < ∞ ,$$

the distance N=N(μ,ν) can be represented as
N=2EL(X,Y)−EL(X,X′)−EL(Y, Y′), Equation (3)
where X′ and Y′ are independent copies of the vectors X and Y, respectively, and E is the symbol of expectation.

Consider the following auxiliary scalar random variables:
U=L(X,Y)−L(X,X), V=L(Y′, Y″)=L(X″, Y″), Equation (4)
where X′, X″ are independent copies of X; the same notation holds for Y. The transformation (4) is introduced to establish a ranking of the points in R^{d} much like the so-called d-functions used for constructing multivariate tolerance regions (see Wilks, S.S. Mathematical Statistics, New York, Wiley (1962)). The simplest way to generate independent copies of X and Y is to split the samples under study into three parts. While this method is not efficient in its use of the data, it illustrates the underlying theoretical principles. Let F_{U} and G_{V} be cumulative distribution functions of the (unobservable) random variables U and V, respectively. The rationale for switching from the vectors X and Y to the scalars U and V is that the null hypothesis H₀: N(μ, ν)=0 is equivalent to H₀* : F_{U}=G_{V} (see Equation (3) for the distance N). The hypothesis H₀* can then be tested using any available distribution-free test, for example, the Kolmogorov-Smimov, Cramér-von Mises, or Mann-Whitney two-sample tests.

The available sample size may not allow splitting the sample into sufficiently many parts to produce independent copies of X and Y. A bootstrap counterpart of the proposed test is desirable to remedy this difficulty. This can be accomplished, for example, by sampling with replacement from x₁, . . . , xₙ and y₁, . . . , yₙ to form the following bootstrap samples
uᵢⱼₗ=L(xᵢ,yᵢ)−L(xᵢ,xⱼ), vᵢⱼₗ=L(yⱼ,y₁)=L(x₁,y₁), i,j,l=1, . . . n.

These values are used to compute the resampling counterparts Fₙ*(z) and Gₙ*(z) of the distribution functions F_{U} and G_{V}. It is clear that Fₙ*(z) and Gₙ*(z) converge to F_{U}(z) and G_{V}(z) when n is large. Thus, a pertinent nonparametric test can be built on the distributions Fₙ*(z) and Gₙ*(z). For example, the Kolmogorov-Smirnov statistic may be defined as κₙ=sup_{z}|Fₙ*(z)−Gₙ*(z) | yielding valid p-values for testing the hypothesis H₀. In another embodiment, the pairs uᵢⱼₗ, vᵢⱼₗ are generated using a subsampling version of the delete-d-jackknife method.

The above described test can be applied to test the hypothesis H₀=Hᵢ for each of the m genes if X and Y are replaced with the corresponding vectors of sample correlation coefficients Rᵢⁿ( [symbol] ) and Rᵢⁿ( [symbol] ). The components of Rᵢⁿ( [symbol] ) and Rᵢⁿ( [symbol] ) are given by Equation (1). Since a total of m hypotheses are to be tested, multiple testing adjustments may be incorporated in the procedure. In exemplary embodiments, both the familywise error rate and the false discovery rate controlling methods can be used for this purpose. A more detailed algorithm is provided below.

It is also possible to use an empirical counterpart of the multivariate N -distance instead of the kernel transformation of Equation (3), using permutations to estimate individual p-values. However, this approach is less preferable for testing the hypothesis Hᵢ because the associated algorithm is computationally intensive, especially if used in conjunction with multiple testing adjustments that require unadjusted p-values as the input.

The presence of a multiplicative technological noise does not present a significant barrier to testing the identical distribution of ihe sample correlation coefficients in two-sample comparisons, at least for Affymetrix oligonucleotide chips for which it may be assumed that there is an identical distribution of random noise in the two samples under comparison. An application of this method to two-color cDNA arrays may require compensation for different distributions of noise in the samples.

Let Aᵢ, Bᵢ, i=1, . . . , m be a pair of independent random variables representing the true expression levels of the i th gene for the two phenotypes under comparison. These variables describe the biological (between-subject) variation and should not be confused with the measurement errors (technological noise) inherent in microarray technology. In addition to this variability a multiplicative array-specific random effect is assumed to be present and caused by the technological noise. Under this model, the observed expression signals Xᵢ and Yᵢ produced by the i th gene are represented as
Xᵢ=αAᵢYᵢ =βBᵢ, i=1, . . . , m
where α and β are positive random variables independent of Aᵢ and Bᵢ. Suppose a total of n random samples of the vectors X=X₁, . . . , Xₘ and Y=Y₁, . . . , Yₘ are available, then
Xᵢⱼ=αⱼAᵢⱼ, Yᵢⱼ=βⱼBᵢⱼ, i=1, . . . , m, j=1, . . . , n
where j is the index of a given array in the pooled sample of size n. The model implies that the same multiplicative measurement error is shared by all genes on each array, but the level of this error varies randomly from array to array.

Generally, the true biological signals Aᵢⱼ, Bᵢⱼ are not recoverable from Xᵢⱼ, Yᵢⱼ without additional assumptions. However, the objective is to test the hypothesis: H₀: Aᵢ [symbol] Bᵢ (Aᵢ and Bᵢ are identically distributed) for the i th gene. In this particular case, the following result will occur: If the random variables aα and β as well as Xᵢ and Yᵢ have finite moments of order r>0. Further, If α [symbol] β, then testing the hypothesis H₀: Aᵢ [symbol] Bᵢ is equivalent to testing the hypothesis H₀′: Xᵢ [symbol] Yᵢ.

These results can be proven as follows. The relationship X [symbol] Y is equivalent to the equality φ_{X}(s)=φ_{Y}(s), where φ_{U}(s)=E {U^{s}} is the Mellin transform. The transforms φ_{X}(s) and φ_{Y}(S) are analytical functions of s for |s|≦r/2. Then the result follows from the uniqueness theorem for the Mellin transform defined as a function of the real variable s on the interval [0, r/2].

This result applies equally to the sample correlation coefficients which are Borel functions of the original expression measurements. It should be understood that the presence of random noise has some effect on the performance of the proposed test.

Since the noise adds variability to the data, it may affect the power of the test, thereby making it more conservative. However, this is a small price to pay for gaining access to critical biological information and can be alleviated by increasing the sample size.

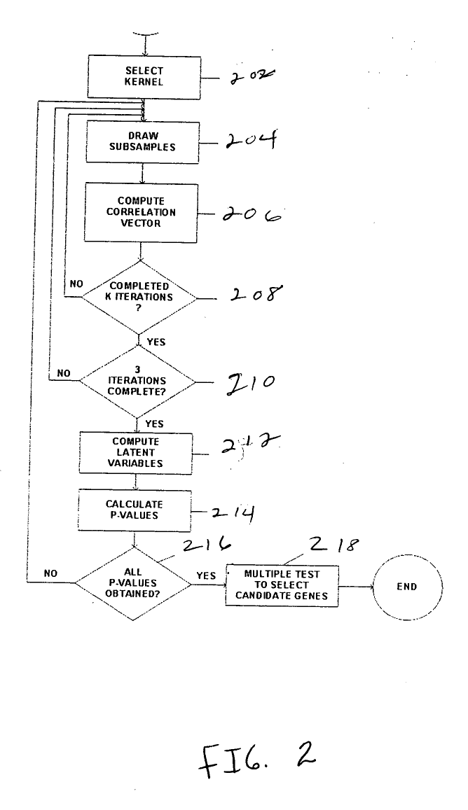

FIG. 2 is a flow chart disclosing a further exemplary algorithm for identifying genes of interest. As shown in FIG. 2, the algorithm begins with selection of a kernel in step 202. Step 202 preferably includes choosing and fixing a specific kernel L(x,y) defined for arbitrary vectors x, y ∈R^{d} with d=m−1. Two useful examples of a kernel L will be provided, although it will be understood that any appropriate kernel can be used within the scope of the invention.

When testing the hypothesis Hᵢ, one option is to use the Euclidean distance between points in R^{d} in the following kernel:

$${L}_{1} ( x , y ) = ‖ x - y ‖ = \sqrt{{\sum }_{k = 1}^{d} {( {x}_{k} - {y}_{k} )}^{2}} . \qquad Equation ( 5 )$$

However, to make the test more sensitive to departures from Hᵢ′, the following kernel is preferred:

$${L}_{2} ( x , y ) = {\sum }_{k = 1}^{d} \vert {x}_{k} - {y}_{k} \vert . \qquad Equation ( 6 )$$

The kernel L₂ given by Equation (6) is a negative definite kernel but not a strictly negative definite one. This means that the N-distance with the kernel L₂ is no longer a metric in the space of all multivariate distributions. However, this distance has the following useful property. Consider the N-distance with the kernel L₂ between two d-variate distributions μ and ν. The N-distance thus defined is equal to zero if and only if all the marginal distributions of μ are identical to the marginal distributions of ν. It follows that, when used in Equation (4), the kernel L₂ makes the test especially sensitive to changes in marginal distributions and hence to departures from the hypothesis Hᵢ′.

Next, in step 204, subsamples are drawn from Groups A and B. In a preferred embodiment, the samples are drawn by first fixing the desired size k≦n of the subsamples. The subsamples may be drawn by the bootstrap (delete-d-jackknife) method. Then, a subsample X₁=Zₗ_{₁}( [symbol] ), . . . , Xₖ=Zₗ_{ₖ}( [symbol] ) of size k is drawn randomly with or without replacement from the original collection of n sample vectors reporting expression levels of the m genes for each of the n subjects in Group [symbol] .

Similarly, a subsample Y₁=Zₛ_{₁}( [symbol] ), . . . , Yₖ=Zₛ_{ₖ}( [symbol] ) is drawn from the original data points pertaining to Group B. The values X₁, . . . , Xₖ and Y₁, . . . , Yₙ are samples from m-dimensional random vectors X and Y, respectively.

In step 206, a correlation vector is computed. In an exemplary embodiment, an integer i is selected such that 1≦i ≦m, and the (m−1)-dimensional vector R₁ [symbol] (i) of the (empirical) correlations (given by Equation (1)) between the i th coordinate of the vector X and all of its other coordinates is computed. A corresponding vector is also constructed from the samples pertaining to Group [symbol] and is denoted by R₁ [symbol] (i).

In step 208, if steps and 206 have not been repeated k times to obtain S₁(i)=R₁ [symbol] (i), . . . , Sₖ(i)=Rₖ [symbol] (i), and T₁(i)=R₁ [symbol] (i), . . . , Tₖ(i)=Rₖ [symbol] (i), control passes to step 204 and the sampling and computation process is thus repeated for k iterations.

In step 210, if steps 204-208 have not been repeated three times, control passes to step 204 two more times to obtain: S₁′(i), . . . , Sₖ′(i), T₁′(i), . . . , Tₖ′(i), S₁′(i), . . . , Sₖ″(i), and T₁″(i), . . . , Tₖ″(i).

In step 212, latent variables are computed. In a preferred embodiment, these variables are calculated using the following formulae:
Uⱼ(i)=L(Sⱼ(i),Tⱼ(i))−L(Sⱼ(i),Sⱼ′(i)) and Vⱼ(i)=L(Tⱼ′(i),Tⱼ(i))−L(Sⱼ″(i),Tⱼ″(i)),j=1, . . . , k.

In step 214, a test is applied to obtain p-values. For example, a distribution-free two-sample univariate test (e.g., the Kolmogorov-Smirnov test) may be applied to the samples Uⱼ(i) and Vⱼ(i), j=1, . . . , k, to obtain the corresponding p-value.

Through an iterative process, as will be seen, in a preferred embodiment all m unadjusted p-values for the hypotheses Hᵢ will be obtained. The resultant p-values are then adjusted for multiple testing.

In step 216, if all m unadjusted p-values for the hypotheses Hᵢ have not yet been obtained, control passes to step 204 and steps 204, 206, 208, 210, 212, and 214 are repeated iteratively for i=1, . . . , m until m unadjusted p-values for the hypotheses Hᵢ have been obtained.

In step 218, a multiple testing procedure is applied, in the manner described above, to finally select the candidate genes of interest.

The additional information provided by the algorithms just described can be used to make a final selection of candidate genes more meaningful. This method can also be used, in combination with existing methodologies, to provide the biologist with an additional source of information for decision making. Through application of the methods disclosed herein, much can be learned about interrelationships between genes and mechanisms by which the cell assigns tasks to different genes to maintain a specific function.

The present invention has been described above with the aid of functional building blocks and method steps illustrating the performance of specified functions and relationships thereof. The boundaries of these functional building blocks and method steps have been arbitrarily defined herein for the convenience of the description. Alternate boundaries can be defined so long as the specified functions and relationships thereof are appropriately performed. Any such alternate boundaries are thus within the scope and spirit of the claimed invention. One skilled in the art will recognize that these functional building blocks can be implemented by discrete components, application specific integrated circuits, processors executing appropriate software and the like or any combination thereof. Thus, the breadth and scope of the present invention should not be limited by any of the above-described exemplary embodiments, but should be defined only in accordance with the claims and their equivalents.

While a preferred embodiment of the present invention has been described above, it should be understood that it has been presented by way of example only, and not limitation. Thus, the breadth and scope of the present invention should not be limited by any of the above-described exemplary embodiments, but should be defined only in accordance with the following claims and their equivalents.

We claim:

1. A method for establishing investigative priorities for genes, the method comprising:
(a) drawing subsamples of vectors from first and second groups of vectors representing expression levels of the genes;
(b) computing a correlation vector of the subsampled vectors in the first group,
(c) computing a correlation vector of the subsampled vectors in the second group,
(d) determining values representing the correlation vectors,
(e) applying a statistical test to the values representing the correlation vectors to obtain p-values;
(f) analyzing the p-values to identify those genes that have a likelihood of changing their relationship with other genes in the sample that exceeds a selected threshold; and
(g) generating an output of results identifying one or more genes that may change their relationship with other genes.

2. The method of claim 1, further comprising using the output in establishing investigative priorities for gene evaluation.

3. The method of claim 1, wherein steps (a) through (c) are performed k times to obtain k correlation vectors, where k>1 is a number of the subsamples.

4. The method of claim 3, wherein said k correlation vectors are obtained three times.

5. The method of claim 4, wherein m unadjusted p-values are obtained, where m>1 is a number of the genes.

6. The method of claim 1, wherein the statistical test is a univariate test.

7. The method of claim 1, wherein step (a) comprises drawing subsamples for a plurality of different phenotypes.

8. The method of claim 7, wherein step (f) comprises identifying those genes whose relationship with other genes varies according to the plurality of different phenotypes.

9. A device for establishing investigative priorities for genes, the device comprising:
an input for receiving first and second groups of vectors representing expression levels of the genes;
a processor, in communication with the input, for:
(a) drawing subsamples of the vectors from the first and second groups of vectors representing the expression levels of the genes;
(b) computing a correlation vector of the subsampled vectors in the first group,
(c) computing a correlation vector of the subsampled vectors in the second group,
(d) determining values representing the correlation vectors,
(e) applying a statistical test to the values representing the correlation vectors to obtain p-values;
(f) analyzing the p-values to identify those genes that have a likelihood of changing their relationship with other genes in the sample that exceeds a selected threshold;
(g) generating an output of results identifying one or more genes that may change their relationship with other genes; and
an output, in communication with the processor, for outputting the output of results generated by the processor.

10. The device of claim 9, wherein the processor performs steps (a) through (c) k times to obtain k correlation vectors, where k>1 is a number of the subsamples.

11. The device of claim 10, wherein said k correlation vectors are obtained three times.

12. The device of claim 11, wherein m unadjusted p-values are obtained, where m>1 is a number of the genes.

13. The device of claim 9, wherein the statistical test is a univariate test.

14. The device of claim 9, wherein the processor performs step (a) by drawing subsamples for a plurality of different phenotypes.

15. The device of claim 14, wherein the processor performs step (f) by identifying those genes whose relationship with other genes varies according to the plurality of different phenotypes.

16. An article of manufacture for establishing investigative priorities for genes, the article of manufacture comprising:
a computer-readable storage medium; and
code, stored on the computer-readable storage medium, the code, when executed on a processor, controlling the processor for:
(a) drawing subsamples of vectors from first and second groups of vectors representing expression levels of the genes;
(b) computing a correlation vector of the subsampled vectors in the first group,
(c) computing a correlation vector of the subsampled vectors in the second group,
(d) determining values representing the correlation vectors,
(e) applying a statistical test to the values representing the correlation vectors to obtain p-values;
(f) analyzing the p-values to identify those genes that have a likelihood of changing their relationship with other genes in the sample that exceeds a selected threshold; and
(g) generating an output of results identifying one or more genes that may change their relationship with other genes.

17. The article of manufacture of claim 16, wherein the code comprises code for controlling the processor to perform steps (a) through (c) k times to obtain k correlation vectors, where k>1 is a number of the subsamples.

18. The article of manufacture of claim 17, wherein the code comprises code for controlling the processor to obtain said k correlation vectors three times.

19. The article of manufacture of claim 18, wherein the code comprises code for controlling the processor to obtain m unadjusted p-values, where m>1 is a number of the genes.

20. The article of manufacture of claim 16, wherein the statistical test is a univariate test.

21. The article of manufacture of claim 16, wherein the code comprises code for controlling the processor to perform step (a) by drawing subsamples for a plurality of different phenotypes.

22. The article of manufacture of claim 21, wherein the code comprises code for controlling the processor to perform step (f) by identifying those genes whose relationship with other genes varies according to the plurality of different phenotypes.
